(19) United States
(12) Patent Application Publication
(10) Pub. No.: US 2005/0278025 A1
(43) Pub. Date: Dec. 15, 2005

(54) Meniscus prosthesis

(75) Inventors: David N. Ku, Atlanta, GA (US); Ralph A. Meyer, Stockbridge, GA (US); Xavier R. Sarabia, Kennesaw, GA (US); Stephen N. Williams, Atlanta, GA (US)

Correspondence Address: JONES DAY, 555 SOUTH FLOWER STREET FIFTIETH FLOOR, LOS ANGELES, CA, 90071, US

(73) Assignee: SaluMedica LLC

(21) Appl. No.: 10/865,238

(22) Filed: Jun. 10, 2004

(51) Int. Cl. A61F 2/30; A61F 2/38

(52) U.S. Cl. 623/14.12; 623/23.41; 623/20.29

(57) ABSTRACT

A prosthesis for placement into a joint space between two or more bones is disclosed. The prosthesis includes a body formed from a pre-formed solid one piece elastomer, wherein the elastomer is formed from a synthetic organic polymer that is biocompatible and has a modulus of elasticity and a mechanical strength between 0.5 MPa and 75 MPa. The body having a shape contoured to fit within a joint space between the femoral condyle, tubercle, and tibial plateau without any means of attachment.

### BACKGROUND OF THE INVENTION

Cartilage may be damaged by direct contact injury, inflammation or most commonly, by osteoarthritis (OA). Osteoarthritis, a process not completely understood by scientists, is the tissue degeneration process that can accompany daily cartilage wear.

Damaged articular cartilage has limited ability to heal due to lack of a direct blood supply. After OA starts, the body can do little by itself to stop tissue deterioration. The injured cartilage goes through a staged degradation process in which the surface softens, flakes and fragments. Finally, the entire cartilage layer is lost and the underlying subchondral bone is exposed. During the early stages, OA symptoms may include stiffness, aching joints and deformity (axial malignment). Because the cartilage layer lacks nerve fibers, patients are often unaware of the severity of the damage. During the final stage, an affected joint consists of bone rubbing against bone, which leads to severe pain and limited mobility. By the time patients seek medical treatment, surgical intervention may be required to alleviate pain and repair the cartilage damage.

A continuum of treatments are available to treat articular cartilage damage in the knee, starting with the most conservative, non-invasive options and ending with total joint replacement if the damage has spread throughout the joint. Currently available treatments, such as anti-inflammatory medications and cartilage repair methods (e.g. arthroscopic debridement) attempt to delay, limit or halt tissue degeneration associated with injury or osteoarthritis. Joint replacement (arthroplasty) is considered as a final solution for older, less active patients when all other options to relieve pain and restore mobility have failed or are no longer effective.

Anti-inflammatory medications manage pain but have limited effect on moderate arthritis symptoms and do nothing to repair joint tissue. One of the most commonly used surgical alternatives—arthroscopic debridement—demonstrates only variable effectiveness at repairing soft tissue. Furthermore, these treatments do not restore joint spacing or contribute to improved joint stability. While knee arthroplasty is effective at relieving pain and restoring stability, the procedure is extremely invasive, technically challenging and may compromise future treatment options.

Consequently, attempts have been made to replace the meniscal cartilage. For example, U.S. Pat. No. 5,171,322 issued to Kenny describes a biocompatible, deformable, flexible, resilient material that is placed in the meniscus and attached to soft tissue surrounding the knee joint; U.S. Pat. No. 5,344,459 issued to Swartz relates to a prosthesis inflatable with air, liquid, or semi-solid; U.S. Pat. No. 6,206,927 and U.S. Pat. No. 6,558,421 issued to Fell teach a meniscus prosthetic device comprising a hard body. However, none of the prior art has been able to achieve a prosthesis capable of providing load distribution properties similar to a human meniscus without the use of attachment means.

### SUMMARY OF THE INVENTION

The present invention relates to a prosthetic device for use in the joint space between two or more bones, more preferably in the joint space between the femoral condyle and the tibial plateau. The device is comprised from an elastomer, wherein the elastomer is formed from an organic polymer that is biocompatible. The elastomer has a modulus of elasticity and a mechanical strength between 0.5 MPa and 75 MPa. The elastic prosthesis can deform to distribute the physiologic loads over a large area such that the joint space is maintained under physiologic loads. The body of the prosthesis has a shape that is contoured to fit with the femoral condyle, the tubercle, and the tibial plateau yet the implant is allowed to translate within the joint space. The device is intended to be used without any means of attachment and remains in the joint space by its geometry and the surrounding soft tissue structures.

It is an object of the invention to provide a cushioning prosthesis for a joint space, in particular a knee joint that is capable of being held in place by its geometry and the surrounding tissue without any additional means of attachment. It is further contemplated that the present invention can provide a cushioning prosthesis for other joint spaces i.e., a temporal-mandibular joint, an ankle, a hip, or a shoulder.

### BRIEF DESCRIPTION OF THE DRAWINGS

FIG. 1 depicts a top plan view of an exemplary medial meniscus prosthesis according to the present invention.

FIG. 2 depicts a perspective anterior-posterior view of an exemplary medial meniscus prosthesis according to the present invention.

FIG. 3 depicts a schematic view of the various regions of an exemplary medial meniscus prosthesis according to the present invention.

FIG. 4 depicts a side view of the cruciate region of an exemplary medial meniscus prosthesis according to the present invention.

FIG. 5 depicts a side view of the outer region of an exemplary medial meniscus prosthesis according to the present invention.

FIG. 6 depicts a perspective view of an exemplary medial meniscus prosthesis according to the present invention implanted in a right knee.

FIG. 7 depicts a top plan view of an exemplary medial meniscus prosthesis according to the present invention seated on a tibial plateau of a right knee.

### DETAILED DESCRIPTION OF THE PREFERRED EMBODIMENT

The present device provides an alternative for those situations in which cartilage degeneration and destruction is present in a single joint compartment. It is an intermediate treatment modality positioned between cartilage repair methods and knee arthroplasty. Rather than debriding soft tissue (menisectomy) or removing and replacing unaffected bone and cartilage (joint replacement), this surgical treatment places a cushioning “spacer” disk into the joint space above the tibial plateau. The device can be placed into the joint space above the tibial plateau. The femur, tubercle, and tibial plateau then articulate against the surface of the device. The device is shaped to conform to the femoral condyle on its superior surface and the tubercle and tibial plateau on its inferior surface and joint capsule on its periphery. The geometric shape of the device further allows for articulation with the femoral condyle, tubercle, and the tibial plateau while keeping the prosthesis in place during knee flexion and extension. The device is intended to be used without any means of attachment and is held in place by its geometry and the surrounding soft tissue structures. As is well known by those skilled in the art, the femoral condyle, tubercle, and tibial plateau of a given knee may vary in shape and size. As such, while various specific shapes are shown and described herein, it should be understood that various other shapes and configurations are contemplated by the present invention.

The device of the present invention is unicompartmental. As used herein, the term “unicompartmental” means that the device is adapted for implantation into a compartment defined by the space between the tibial plateau and a femoral condyle. Thus, the device is suited for use in either a lateral compartment or a medial compartment. Where it is necessary to replace menisci in both compartments, two devices according to the present invention could be used.

The device is made from polymer and saline forming an elastomer that is processed to high strength tolerances. The elastomer further being compliant, wear-resistant, and having load distribution capabilities similar to native articular cartilage and meniscus.

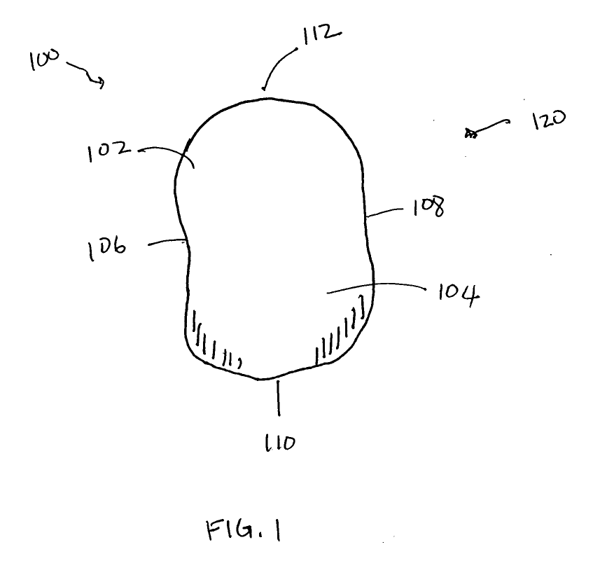

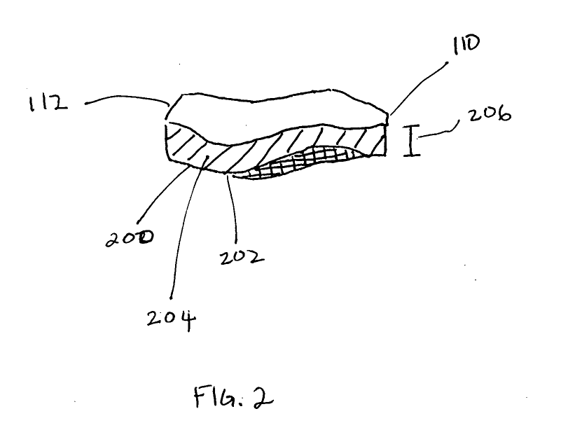

Turning to FIG. 1 and FIG. 2, a prosthesis 100, generally elliptical in shape, comprising a body 120 formed from an elastomer is shown. The elastomer is preferably a pre-formed solid one piece elastomer. In a preferred embodiment, the prosthesis 100 is reniform i.e., kidney shaped. However, other shapes may be used and are contemplated. In particular, the body may be toroidal, circular, planar, donut shaped or crescent shaped.

The prosthesis 100 illustrated in FIG. 1 is intended for use in a medial compartment of a right knee. It should be understood by those skilled in the art that a device according to the present invention for use in the medial compartment of a left knee is simply a mirror image of the device illustrated in FIG. 1.

The elastomer has a modulus of elasticity of less than 75 MPa and a mechanical strength of greater than 0.5 MPa. More preferably, the elastomer has a compressive modulus between 5 and 10 MPa and a tensile strength between 5 and 12 MPa. More preferably, the elastomeric device may be viscoelastic. The body 120 of the prosthesis 100 has a superior surface 102, an inferior surface 200, and an outer wall 204 having a thickness 206 therebetween. The superior surface 102 forms a concave groove channel 104 that is contoured to fit with a femoral condyle while the inferior surface 200 forms a generally convex surface 202 contoured to fit on top of a tibial plateau. The body further includes a cruciate region 106, an outer region 108, an anterior region 110, a posterior region 112 and a central region 114. The outer wall 204 is formed from the periphery of the cruciate region 106, outer region 108, anterior region 110, and posterior region 112.

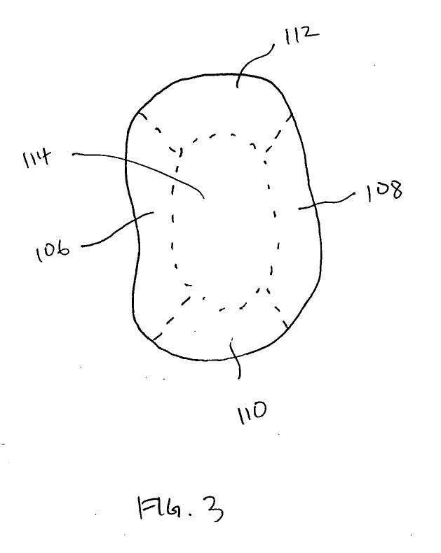

For purposes of illustration only, FIG. 3 generally depicts the relationship between the various regions of the device of the present invention. The posterior region 112 is generally between and contiguous to the cruciate region 106 and the outer region 108. The outer region 108 is generally between and contiguous to the anterior region 110 and the posterior region 112. The anterior region 110 is generally between and contiguous to the outer region 108. The cruciate region 106 is generally between and contiguous to the anterior region 110 and the posterior region 112. The central region 114 is generally between and contiguous to each of the cruciate region 106, outer region 108, anterior region 110, and posterior region 112. It will be noted that the various regions are contiguous and are not capable of being clearly delineated. Instead, the regions are defined merely to provide a point of reference for various aspects of the present invention.

Preferably, the groove channel 104 is located within the central region 114. The groove channel 104 forms a concave surface that rises up to meet the outer wall 204. The concave surface of the groove channel 104 enables the prosthesis 100 to receive the contoured surface of the femoral condyle.

The prosthesis 100 is wide enough to fully receive the width of the femoral condyle. Preferably, the groove channel 104 also has a width that is greater than ½ the width of the body 120. The width is measured from the outer wall 204 of the cruciate region 106 to the outer wall 204 of the outer region 108. The length of the prosthesis 100 is also shown to be approximately the anterior-posterior length of the tibial plateau. By being wider, the prosthesis 100 is able to provide a channel to guide the femoral condyle, aiding the prosthesis 100 to maintain its position within the space between two bones (“joint space”) during kinematic joint motion of the knee. By simultaneously having a generally convex inferior surface 202 that is contoured to sit on top of the tibial plateau, the prosthesis 100 is provided to maintain its position within the joint space with an elastic body 120. Although specific embodiments are described in detail herein, it should be understood that other variations are contemplated by the present invention.

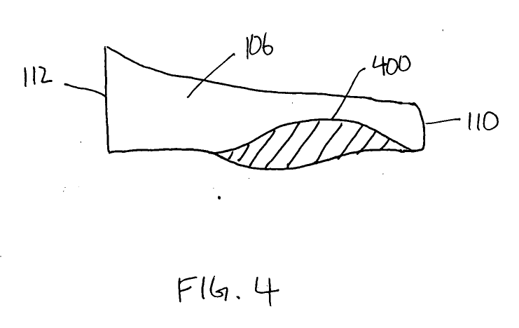

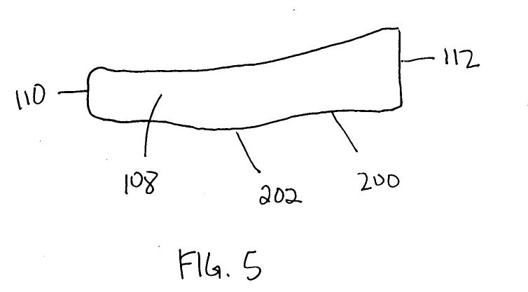

As shown in FIG. 4, the cruciate region 106 contains an indention 400. The indentation 400 is located proximally to the anterior region 110 and decreases in size as it extends from the outer wall 204 of the cruciate region 106 towards the central region 114. Viewing the outer wall 204 of the cruciate region 106, the indentation 400 is generally in the form of a sinusoidal shaped arch. The indentation 400 enables the prosthesis 100 to form a better fit within the joint space by being contoured to fit with the tubercle of the tibia. In contrast FIG. 5 shows the outer region 108 without any indentations.

While a secure fit within the joint space is important, it should be understood that the prosthesis 100 may shift slightly or translate during movement of the joint. In relation to the knee joint, the prosthesis 100 must be able to engage in natural motion, including flexion and extension motions commonly associated with typical movement, without unrecoverably unseating from the tibial plateau. As used herein, “unrecoverably unseating” refers to a shift in the positioning of the device that is so significant that it is unable to return to its original position.

As can be seen from FIG. 5, the posterior region 112 has a greater thickness than the anterior region 110. The greater thickness of the prosthesis 100 at its posterior region 112 aids the prosthesis 100 to stay in place by forming a barrier to anterior displacement through the joint space. The greater thickness of the posterior region 112, however, does not pose a problem during insertion due to the compliant nature of the elastomer. If the thickness of the posterior region 112 is greater than the space between the femoral condyle and the tibial plateau, the prosthesis 100 may be flexed or bent into place. Preferably, the thickness of the posterior region 112 ranges between 3 and 20 mm while the anterior region 110 ranges between 3 and 20 mm. The cruciate region 106, the outer region 108, and the central region 114 have varying thicknesses ranging from 3 and 20 mm.

In another embodiment, the anterior region 110 may be thicker than the posterior region 112. In yet another embodiment, the central region 114 may have a thickness 206 that is equal to or less than the thickness 206 of the cruciate region 106, outer region 108, anterior region 110, or the posterior region 112.

A prosthesis 100 according to the present invention may include one or more sloped areas in the various regions and surfaces to enable the prosthesis 100 to be maintained on the tibial plateau during flexion and extension without the need for any additional securing means. Specifically, the geometry of the prosthesis 100 is selected to enable the body 120 to securely fit between the tibial plateau and the femoral condyle while taking into account the tubercle without the need for cement, pinning, or other surgical securement means.

In a preferred embodiment, the prosthesis 100 has a discoid shape with an anterior to posterior (A-P) length of 38-58 mm. However, additional A-P lengths between 30 and 80 mm are contemplated and may be made available for the specific needs of the patient. The thickness 206 of the prosthesis 100 may vary but are typically between 1-20 mm at any point. However, thickness outside this range is contemplated and may be used depending upon the specific needs of the patient.

While it is envisioned that the prosthesis 100 of this invention will not require a means of attachment beyond its geometry, a tissue fixation component may be combined with the prosthesis 100 to enhance tissue fixation. The tissue fixation component may be comprised of tabs or holes to allow the surgeon to suture the prosthesis 100 to native body structures. Alternatively, the surface roughness and porosity of certain areas of the prosthesis 100 may be tailored to allow for fibrotic in-growth and mechanical interlock. In another embodiment of the present invention, the material may include a biologically active agent that enhances attachment. In yet another embodiment of the present invention, a second material such as polyethylene may be molded in selective areas on the prosthesis 100 to create fibrotic in-growth and mechanical interlock. For example, the tissue fixation component may be in the form of a piece of Dacron® or polyester mesh that can be placed on the surface of the prosthesis 100 to promote adhesion to the tibia or one or more bones of the joint or the joint capsule. Other methods may be used singly or in combination to achieve optimal attachment and these are anticipated. These materials may be calcium granules, fibers, thread, or mesh that are molded into the body of the device. Example materials for tissue fixation or reinforcement include polyester, polyethylene, KEVLAR®, poly-paraphenylene terephthalamide, or other polymer materials, or titanium, tungsten, tantalum, stainless steel, cobalt chromium, or other metal materials that are biocompatible and flexible.

The materials for tissue fixation or reinforcement are molded into the body 120 of the prosthesis 100 during the manufacturing of the part. The material may be completely encapsulated by the elastomer or adherent to the periphery of the prosthesis 100. This reinforcing material may be used to enhance the tensile strength and compressive modulus of the device without providing for tissue fixation. Alternatively, the material may provide for tissue fixation without reinforcement of the ultimate tensile strength.

A device according to any of various aspects of the present invention may be formed from any suitable material that is biocompatible. Preferably, the elastomer or polymeric material is formed from an organic polymer. The polymeric material may further be formed synthetically. More preferably, the polymeric material is selected to have properties that closely resemble those of a native meniscus. According to one variation of the present invention, the device is formed from a biocompatible polymeric material. Suitable materials are generally strong, hydrophilic, biostable, compliant, and have a low coefficient of friction.

In particular, the polymeric material used for the device of the present invention preferably has a uniform modulus of elasticity of from about 0.5 MPa to about 75 MPa. In some instances, the polymeric material may have a uniform modulus of elasticity of from about 1 MPa to about 10 MPa. In yet other instances, the polymeric material may have a uniform modulus of elasticity of from about 2 MPa to about 5 MPa. The polymeric material further enables the body 120 to have cushioning and load distribution capabilities within a joint space similar to native articular cartilage and meniscus.

Another variation of the present invention allows for a non-uniform modulus of elasticity within the part. In particular, the polymeric material used for the device of the present invention may have a stiffer modulus of elasticity along the periphery and a softer modulus of elasticity along the central region 114. Variations in the modulus of elasticity within the range of about 0.5 MPa to about 75 MPa are contemplated and may be used depending upon the specific needs of the patient.

The polymeric material that forms the device of the present invention must be sufficiently strong to withstand repeated stresses caused during typical knee movement. Preferably, the polymeric material has an ultimate tensile strength of from about 0.5 MPa to about 75 MPa. In some instances, the polymeric material may have an ultimate tensile strength of from about 0.6 MPa to about 10 MPa. In yet other instances, the polymeric material may have an ultimate tensile strength of from about 2 MPa to about 8 MPa.

Furthermore, the polymeric material used to form the device of the present invention must have a sufficiently low coefficient of friction to enable the device to move within the meniscal compartment and withstand the repeated motion of the femoral condyle on the superior surface. Specifically, the coefficient of friction must be sufficiently low such that upon flexion and extension motions, the stress on the device created by the femoral condyle does not cause the device to unrecoverably unseat from the tibial plateau. In some instances, the polymeric material may have a dynamic coefficient of friction of from about 0.01 to about 1. In other instances, the polymeric material may have a dynamic coefficient of friction of about 0.02 to about 0.1 against cartilage or roughened bone.

The prosthesis 100 is made from a polymeric material that is comprised of a poly(vinyl alcohol) (“PVA”) and water. The process involves mixing water with PVA crystal to obtain a PVA hydrogel. The PVA hydrogel is then frozen and thawed at least once to create an interlocking mesh between the PVA molecules to create a PVA cryogel. The freezing and thawing may then be repeated many times to obtain the optimal balance between strength and elasticity. Preferably, the prosthesis 100 has an ultimate strength of at least 1 MPa enabling the prosthesis to withstand normal stress loading forces for 10 million cycles typical of those experienced by human knee cartilage. Further information about the PVA is set forth in the applicant's U.S. Pat. No. 6,231,605 B1, dated May 15, 2001, issued to Ku for “Poly(Vinyl Alcohol) Hydrogel and U.S. Pat. No. 5,981,826, dated Nov. 9, 1999, issued to Ku for “Poly(Vinly Alcohol) Cryogel,” each of which are incorporated herein by this reference in its entirety.

The device according to the various aspects of the present invention may be used in conjunction with biologically active substances. Many such bioactive agents would be released gradually from the material after implantation, and thereby delivered in vivo at a controlled, gradual rate. The device may thus be used as a drug delivery vehicle.

Some bioactive agents may be incorporated into the device to support cellular growth and proliferation on the surface of the material. Bioactive agents that may be included in the replacement include, for example, growth factors, anti-inflammatory drugs, antibodies, cytokines, integrins, monoclonal antibodies, proteins, proteases, anticoagulants, and glycosaminoglycans.

The prosthesis 100 may be implanted using standard orthopedic surgery techniques. Prior to use, it must be confirmed that the ligamentous structures in the knee are intact. This can be done using a variety of methods. One in particular that is noninvasive is magnetic resonance imaging (MRI).

Once the indications are confirmed, osteophytes from the femoral condyle and tibial plateau are removed, allowing the collateral ligament to regain its normal movement.

Implantation of the prosthesis 100 may be performed using existing surgical techniques. The implantation process may be improved by developing instrumentation to facilitate sizing, insertion and removal. Sizing could be determined more efficiently using a length gauge to measure the A-P length of the tibial plateau. The length gauge would have an atraumatic means of locating the distal portion of the tibial plateau. By locating the distal portion of the tibial plateau as a reference point, the gauge could extend until the proximal surface of the tibial plateau was traversed. The distance between the displacement would correspond to one of the sizes of the prosthesis 100. Similarly, thickness gauges could be used to determine the appropriate size of prosthesis 100 to implant. To facilitate insertion and removal of the prosthesis 100, an atraumatic clamp with non-cutting edges could be used. The atraumatic clamp would have blunt surfaces to allow the instrument to be inserted between the surfaces of the prosthesis 100 and cartilage, and grip the slippery prosthetic without damaging the device.

Then the meniscus is resected in its entirety, ensuring that the circumferential fibers of the posterior horn have been fully disconnected from the posterior horn insertion, as any connected fibers may lead to poor device seating or to dislocation. Using a rasp, burr or curette, irregularities in the femoral and tibial articular surfaces are removed.

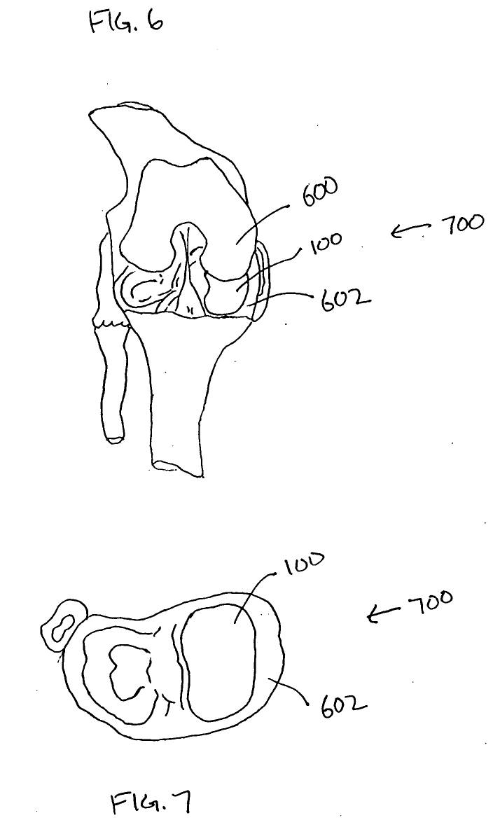

FIG. 6 illustrates an appropriately sized prosthesis 100 having an appropriate thickness and an A-P length that is approximately equal to or slightly longer than the dimensions of the joint space being inserted between the tibia and the femur. Starting with the appropriate A-P length and thickness, place the prosthesis 100 starting with the knee in flexion and external rotation and applying pressure to the prosthesis 100 as the knee is slowly extended and internally rotated. As is well known by those skilled in the art, the space between the medial condyle 600 and the tibial plateau 602 may vary in dimension depending on a variety of factors. Thus, the prosthesis 100 of the present invention may be formed to have any suitable thickness 206. In some instances, the thickness 206 of the prosthesis 100 may be adapted to fit within a relatively small gap of less than about 3 mm. In other instances, the thickness 206 of the prosthesis 100 may be adapted to fit within a gap from about 3 to about 6.5 mm. In yet other instances, the thickness of the prosthesis 100 may be adapted to fit within a relatively large gap of greater than 6.5 mm. While specific gap dimensions are provided herein, it should be understood that the prosthesis 100 of the present invention may be adapted to accommodate a variety of gap sizes as needed.

Again referring to FIG. 6, the prosthesis 100, is inserted concave side up, at an initial 45 degree angle to the tibial spine with the posterior region 112 leading the insertion. The indentation 400 of the prosthesis 100 should be on the cruciate region 106, and the posterior region 112 of the prosthesis is thicker than the anterior region 110. Lateral, rotational pressure is applied to the prosthesis 100 while slowly extending the knee.

FIG. 7 depicts a top plan view of the prosthesis 100 seated on the tibial plateau 602 of a right knee 700. The prosthesis 100 generally occupies the same or similar area that would be occupied by a natural medial meniscus (not shown). As is well known by those skilled in the art, the medial compartment may vary in dimension depending on the age and bone structure of the subject knee 700. As such, the prosthesis 100 may be adapted to accommodate various sizes of a knee 700. In some instances, the anterior to posterior length (as measured from the most distal points of the two regions) of the device may be from about 30 to about 70 mm. In some instances, the anterior-to-posterior length may be from about 30 to about 75 mm. In some specific instances, the anterior-to-posterior length may be about 38-58 mm. While specific anterior-to-posterior lengths are provided herein, it should be understood that other anterior-to-posterior lengths are contemplated by the present invention.

When the prosthesis 100 moves into position, the knee is manipulated through several flexion-extension cycles. The prosthesis 100 is appropriately sized if it stays in position without limiting full range of motion throughout multiple flexion-extension cycles. Once in position, the femur articulates with the superior surface 102. The device is intended to be used without cement and is held in place by compatible geometry and surrounding soft tissue structures. The knee should be stable at full extension. Ligaments should not be overstretched with the prosthesis 100 in place at any phase of the flexion-extension cycle. Anterior-posterior translation of the prosthesis 100 is normal. The prosthesis 100 should track the femur throughout the flexion-extension cycle. Finally, closure is effected using standard operative techniques.

Unlike other prosthesis, after implantation, the joint area may be viewed using an Magnetic Resonance Imager (MRI) since the prosthesis 100 does not contain metal parts which would cause interference. The invention described includes the development of orthopaedic devices that have a good MRI signature and does not distort the image in the surrounding tissue. The device may also contain radio-opaque markers to better locate the part with X-ray images.

It is readily apparent to those skilled in the art that numerous modifications, alterations, and changes can be made without departing from the inventive concept described herein. The invention, therefore, is not to be restricted except in the spirit of the appended claims.

1. A prosthesis comprising a body formed from an elastomer, wherein the elastomer is formed from an organic polymer that is biocompatible and has a modulus of elasticity and a mechanical strength between 0.5 MPa and 75 MPa enabling said body to provide cushioning and load distribution capabilities within a joint space similar to native articular cartilage and meniscus, said body having a shape contoured to fit with a femoral condyle, a tubercle, and a tibial plateau, said body having a geometry designed to stay within a joint space without any separate means of attachment.

2. The prosthesis of claim 1, wherein the body has a uniform modulus of elasticity throughout.

3. The prosthesis of claim 1, wherein the body includes a superior surface forming a concave groove channel contoured to receive a femoral condyle, an inferior surface forming a convex surface contoured to fit on top a tibial plateau, and having a thickness therebetween.

4. The prosthesis of claim 3, wherein the groove channel has a width that is greater than ½ the width of the body.

5. The prosthesis of claim 1, wherein the body further comprising a cruciate region, an outer region, an anterior region, a posterior region a central region, and an outer wall along the periphery of the cruciate region, outer region, anterior region, and posterior region, said cruciate region including an indentation located proximally to the anterior region and contoured to receive a tubercle.

6. The prosthesis of claim 5, wherein the indentation is generally in the form of a sinusoidal shaped arch.

7. The prosthesis of claim 5, wherein the indentation decreases in size as it extends from the outer wall of the cruciate region to the central region.

8. The prosthesis of claim 3, wherein the groove channel is located within a central region.

9. The prosthesis of claim 1, wherein the body is wide enough to fully receive the width of a femoral condyle.

10. The prosthesis of claim 1, wherein the body has a length that is approximately equal to the anterior-posterior length of a tibial plateau.

11. The prosthesis of claim 5, wherein the posterior region is thicker than the anterior region.

12. The prosthesis of claim 1, wherein the body may be flexed into a joint space.

13. The prosthesis of claim 1, wherein the body is compatible with magnetic resonance imaging.

14. The prosthesis of claim 1, wherein the body is attached to a tissue fixation component.

15. The prosthesis of claim 14, wherein the tissue fixation component is selected from the group consisting of extension tabs, sutures, and mesh.

16. The prosthesis of claim 1, wherein the body is substantially kidney shaped.

17. The prosthesis of claim 1, wherein the body is substantially toroidal in shape.

18. The prosthesis of claim 1, wherein the body is substantially crescent shaped.

19. The prosthesis of claim 5, wherein the anterior region is thicker than the posterior region.

20. The prosthesis of claim 1, wherein the body includes a reinforcing material selected from the group consisting of polymers such as polyester or metals such as titanium.

21. The prosthesis of claim 1, wherein the elastomer is a hydrogel.

22. The prosthesis of claim 1, wherein the elastomer has a modulus of elasticity of from about 0.6 MPa to about 10 MPa.

23. The prosthesis of claim 1, wherein the elastomer has a compressive modulus of elasticity of from about 2 MPa to about 5 MPa.

24. The prosthesis of claim 1, wherein the elastomer has a tensile strength of from about 0.6 MPa to about 10 MPa.

25. The prosthesis of claim 1, wherein the elastomer has a tensile strength of from about 2 MPa to about 5 MPa.

26. The prosthesis of claim 1, wherein the elastomer has a dynamic coefficient of friction from about 0.01 to about 1.

27. The prosthesis of claim 1, wherein the elastomer has a dynamic coefficient of friction from about 0.02 to about 0.1.

28. The prosthesis of claim 1, wherein the elastomer is formed synthetically.

29. The prosthesis of claim 1, wherein the body is formed from a pre-formed solid one piece elastomer.

30. A prosthesis comprising a body formed from a pre-formed solid one piece elastomer, wherein the elastomer is formed from an organic polymer that is biocompatible and has a modulus of elasticity and a mechanical strength between 0.5 MPa and 75 MPa enabling said body to provide cushioning and load distribution capabilities within a joint space similar to native articular cartilage and meniscus, said body including a superior surface forming a concave groove channel contoured to receive a femoral condyle, wherein said groove channel has a width that is greater than ½ the width of the body, an inferior surface forming a convex surface contoured to fit on top of a tibial plateau, and having a thickness therebetween, said body being wide enough to fully receive the width of the femoral condyle, said body further comprising a cruciate region, an outer region, an anterior region, a posterior region, a central region, and an outer wall surrounding the periphery of the cruciate region, outer region, anterior region, and posterior region, said cruciate region including an indentation located proximally to the anterior region and contoured to fit with a tubercle, said indentation generally in the form of a sinusoidal shaped arch decreasing in size as it extends from the outer wall of the cruciate region to the central region, said posterior region being thicker than said anterior region, said body further having a geometry designed to stay within a joint space without any separate means of attachment.

31. A method for placing a prosthesis into a joint space which comprises:
making an incision in the tissue surrounding the joint space of a knee;
inserting a prosthesis into the joint space of a knee, said prosthesis comprising a body formed from a pre-formed solid one piece elastomer, wherein the elastomer is formed from an organic polymer that is biocompatible and has a modulus of elasticity and a mechanical strength between 0.5 MPa and 75 MPa, said body including a superior surface forming a concave groove channel contoured to receive a femoral condyle, wherein said groove channel has a width that is greater than ½ the width of the body, an inferior surface forming a convex surface contoured to fit on top of a tibial plateau, and having a thickness therebetween, said body being wide enough to fully receive the width of the femoral condyle, said body further comprising a cruciate region, an outer region, an anterior region, a posterior region, a central region, and an outer wall surrounding the periphery of the cruciate region, outer region, anterior region, and posterior region, said cruciate region further including an indentation located proximally to the anterior region and contoured to fit with a tubercle, said indentation generally in the form of a sinusoidal shaped arch decreasing in size as it goes from the outer wall of the cruciate region to the central region, said posterior region being thicker than said anterior region, said body further having a geometry designed to stay within a joint space without any separate means of attachment; and
closing said incision.
